(19) United States
(12) Patent Application Publication
(10) Pub. No.: US 2007/0042052 A1
(43) Pub. Date: Feb. 22, 2007

(54) Inorganic antibacterial agents containing high valent silver and preparation method thereof

(76) Inventors: Jian Zhao, Shanxi (CN); Wenzheng Zhang, Shanxi (CN); Guangwen Wang, Shanxi (CN)

Correspondence Address: Raymond Y Chan, 108 N Ynez Avenue, Suite 128, Montery Park, CA, 91754, US

(21) Appl. No.: 10/578,532

(22) PCT Filed: Oct. 27, 2004
(86) PCT No.: PCT/CN04/01218

§ 371 Date: May 2, 2006

(30) Foreign Application Priority Data
Nov. 4, 2003 (CN) 200310105878.3

(51) Int. Cl. A61K 33/38

(52) U.S. Cl. 424/618

(57) ABSTRACT

The present invention provides inorganic antibacterial agents containing high valent silver, which are characterized by containing 2-6% by weight divalent silver, trivalent silver or tetravalent silver based on the total weight of the antibacterial agents, wherein said high valent silver is introduced onto the solid carriers by ion exchanges.

### BACKGROUND OF THE PRESENT INVENTION

1. Field of Invention

The present invention relates to solid inorganic antibacterial agents containing high valence silver and preparation method thereof, and more importantly, relates to solid inorganic antibacterial agents containing divalent silver, trivalent silver or tetravalent silver, said inorganic antibacterial agents with high valence silver can be broadly applied in antibacterial plastic products, antibacterial fiber products, antibacterial clothing products, antibacterial coating products, and antibacterial sanitary products and etc.

2. Description of Related Arts

Silver ion, copper ion and zinc ion all have been effective in antibacterial, antifungal, and antivirus applications. Silver ion has the highest antibacterial activities among all the metallic ions. Nowadays, the silver-contained inorganic antibacterial agents have been widely used in many fields, such as antibacterial plastic products, antibacterial clothing, antibacterial daily and home electronic appliances, antibacterial sporting products, antibacterial medical products, and antibacterial construction materials.

There are a variety of inorganic silver-contained antibacterial agents available in practices. For examples, U.S. Pat. Nos. 4,911,898 and 4,938,958 disclosed the techniques for carrying sliver zeolite. U.S. Pat. Nos. 5,296,238 and 5,441,717 disclose the techniques of silver contained inorganic zirconate phosphate antibacterial agents, such as Ag_{0.16}Na_{0.84}Zr₂(PO₄)₃, Ag_{0.05}H_{0.05}Na_{0.90}Zr₂(PO₄)₂ and etc. The above mentioned silver is unexceptionally embodied as monovalent sliver ion being exchanged with Na⁺ and afterwards supported onto the zeolite carrier or zirconate phosphate carrier.

Japanese patents 6-263612 and 6-263613 use silver-contained antibacterial agents, such as silver-contained zirconate phosphate, (and silver-contained stannum, phosphate, and silver-contained titanium phosphat), dissolved within the organic solvent to be grinded by zirconia sphere under a dispersant agent so as to increase its antibacterial activity.

JP2000-68914 discovers the use of applying acetic acid into inorganic antibacterial agents to increase its antibacterial performance.

Nevertheless, the antibacterial agents described above is unexceptionally monovalent silver antibactterial agents. On the other hand, the antibacterial performance of silver ions is correlated to its valence form. The preference of the antibacterial performance of the silver ions is: Ag³⁺>Ag²⁺>Ag¹⁺. Although antibacterial performance of the different silver valence would be varied in practices for treating heterogeneous bacteria, it is well understood that divalent silver's antibacterial performance is about 50-250 times better than monovalent silver in general. Below shows the potentiometric mensuration of Ag¹⁺, Ag²⁺, and Ag³⁺.
Ag¹⁺+e→Ag 0.7994 ev
Ag²⁺+2e→Ag 2.58 ev
Ag³⁺+3e→Ag 3.36 ev

Sliver oxide Ag₂O could be converted to silver peroxide Ag₂O₂ after being treated with the strong oxidant. Silver peroxide consists of one trivalent silver ion and one monovalent silver ion, such as Ag—O—Ag=O, and it has a higher antibacterial performance against Ag₂O. In case of such silver peroxide is dissolved into concentrated acids, such as nitric acid, sulfuric acid, perchloric acid, phosphoric acid, Ag³⁺ and Ag¹⁺ ions can be immediately generated. Afterwards, trough the below formulas, Ag³⁺ and Ag¹⁺ could be converted to stablize Ag²⁺ in acidic solvent.
Ag¹⁺−e=Ag²⁺
Ag³⁺+e=Ag²⁺

It is widely known that divalent silver has higher antibacterial performance than monovalent silver. USS, 017,295 discloses antibacterial agents containing divalent silver. However, such divalent sliver will be only kept stable in concentrated acidic environment. As a result, it would be rather difficult and dangerous for the operation, usage, and transportation of such agents.

U.S. Pat. No. 5,089,275 provides a type of solid antibacterial compound containing divalent silver. This compound is prepared through reacting acidic fluid divalent silver complex with anhydrous calcium sulfate so as to obtain stable hydrated solid. Although the solid antibacterial agents containing divalent silver solves the issue of the liquid state of divalent silver antibacterial agents, the product still faces the deficiency of long term storage stability because divalent silver is not supported onto the solid carriers by ion exchange. Therefore, the field of application is limited due to the fact to its water solubility. i.e. such solid antibacterial agents have to be used in cleaning water, such as swimming pool, bathtub, industry cooling system, and so on.

Therefore, it is desirable to make intensified investigate into solid inorganic antibacterial agents containing divalent silver to broaden its field of applications.

### SUMMARY OF THE PRESENT INVENTION

A primary object of the present invention is to provide an inorganic antibacterial agent containing high-valence silver, which is characterized by containing 2 to 6% by weight divalent silver, trivalent silver or tetravalent silver based on total weight of the antibacterial agents, wherein the said high-valence silver is-supported onto a solid carrier by ion exchange reaction.

Another object of the present invention is to provide a method for preparing an inorganic antibacterial agent containing high-valence silver, comprising the following steps: adding a solid carrier, which is capable of ion exchange, into a solution containing the high-valence silver, wherein the high-valence silver solution has a divalent silver concentration of 2-8% in weight, preferably 3.5-5% in weight; substantially stirring the solution to obtain a pulp formed solution for enabling an ion exchange reaction between the high-valence silver ion and the exchangeable ion of the solid carrier to yield solid compound, filtering and drying the solid compound to ultimately obtain the inorganic antibacterial agent containing the high valence silver.

Another object of the present invention is to provide a plurality of manufactures applying the inorganic antibacterial agents containing high-valence silver, such as applications in antibacterial clothing, antibacterial daily products, antibacterial plastic products, antibacterial medical and mechanical devices, antibacterial construction materials, antibacterial ceramics, antibacterial sanitary utensils, and antibacterial home electronic appliances.

These and other objectives, features, and advantages of the present invention will become apparent from the following detailed description, the accompanying drawings, and the appended claims.

### BRIEF DESCRIPTION OF THE DRAWINGS

FIG. 1 is a XPS energy spectrum of the inorganic antibacterial agent containing monovalent silver and divalent silver

### DETAILED DESCRIPTION OF THE PREFERRED EMBODIMENT

According to the present invention, the contained high valence silver weight percentage in the inorganic antibacterial agents is defined between 2 to 6%, preferably 2 to 5%, best at 3.7% by weight of divalent silver, trivalent silver or tetravalent silver, wherein the high valence silver is introduced onto the solid carriers by ion exchange. Average diameter of the inorganic antibacterial agents is 1.0-10.0 μm, preferably 1.0-2.0 μm.

The preferred carriers which are capable of ion exchange are selected from a group consisting of sodium zirconium phosphate, sodium titanium phosphate, sodium tin phosphate and zeolite. Wherein zeolite are A type zeolite, X type zeolite, or Y type zeolite.

Accordingly, the present invention further introduces a method for preparing an inorganic antibacterial agent containing high-valence silver, wherein the method comprises the following steps: adding a solid carrier, which is capable of ion exchange, into a solution containing the high-valence silver substantially stirring the solution to obtain a pulp formed solution for enabling an ion exchange reaction between the high-valence silver ion and the exchangeable ion of the solid carrier to yield solid compound, filtering and drying the solid compound to ultimately obtain the inorganic antibacterial agent containing the high valence silver.

In the above mentioned method, the high valence silver solution are formed from by dissolving silver peroxide into persulphate or concentrated nitric acid to generate water solution containing bivalent sliver, periodic acid solution containing trivalent silver, and sliver acid solution containing tetravalent silver.

The volume ratio between the solid carriers to the high valence silver solution is 1:6-10, preferably 1:8. The environment for ion exchange reaction between the carriers and the high-valence silver is pH 1-5, preferably 3-3.5, 30° C.-80° C. in temperature, preferably 55° C.-65° C., best at 60° C. The reaction takes 2-8 hours, preferably 4-6 hours, and best at 6 hours. 20% of NaOH or KOH are used to adjust the system's pH.

Accordingly to the present invention, the filtering and drying step further comprises sub-steps for washing a filter cake until the pH value ranged between 5-6, preferably 6, and for drying the filter cake at a temperature between 110° C.-140° C., preferably at 120° C. for 1-2 hours, and calcinating the filter cake between 800° C. to 1000° C., preferable at 900° C., for 2-4 hours, preferable 2 hours, and then grinding the filter cake by a gas flow pulverizer to obtain particles with a size of average diameter of 1.0-10.0 μm, preferably 1.0-2.0 μm.

According to the present invention, bivalent silver ions is adapted to reacted with ion-exchangers such as sodium zirconium phosphate, sodium titanium phosphate, and sodium tin phosphate to exchange a portion of Na ions such as from NaZr₂(PO₄)₃, preferably to exchange 10%-60% of Na ions, best at 30%. It is also able to exchange Na ions from either A type zeolite, X type zeolite, or Y type zeolite, so that the divalent silver can be supported onto the solid carriers to prepare inorganic antibacterial agents containing divalent silver. Accordingly, one can use the same method, to support trivalent sliver such as silver periodate or tetravalent silver such as silver acid onto the solid carriers such as sodium zirconium phosphate or zeolite to prepare inorganic antibacterial agents containing even higher valence silver.

Additionally, divalent silver compound is capable of reacting with phosphate to generate bivalent sliver phosphate or reacting with molybdate to generate AgOMoO₃ inorganic antibacterial agents.

The following descriptions of embodiments explain applications of the present invention. It should be understood that the scope of invention is not limited to the following embodiments only.

### EXAMPLE 1

800 ml of de-ionized water and 0.026 mol of potassium persulfate (6.94 g) are added into a 1000 ml three-necks bottle comprising a stirrer and a controller. While stirring up, 0.017 mol silver peroxide (4.25 g) is added to the mixture solution until the silver peroxide completely dissolved. And then, 100 g of [NaZr₂(PO₄)₃] (zirconium sodium phosphate) is added to the mixture solution, and 20% of sodium hydroxide is used to adjust the mixture solution's pH value to a range 3-3.5. After then, the solution is heated to reach 60° C. to react for 6 hours. The mixture solution is then cooled at room temperature. Afterwards, the solution is filtered and the filter cake is rinsed until the pH value hit 6, and then the filter cake is dried at 120° C. for 1 hour and then be calcined at 900° C. for 2 hours, finally, the calcined substance is grinded by an air flow pulverizer to obtain powders having an average diameter 2.0 μm and 100 g of 3.7% in weight of silver-contained zirconium phosphate inorganic antibacterial agents.

### EXAMPLE 2

The preparing method is same with the above example 1, wherein 100 g of zirconium sodium phosphate is replaced by 100 g A-type zeolite to ultimately prepare 100 g of 3.7% in weight of silver-contained inorganic antibacterial agents.

### EXAMPLE 3

800 ml of de-ionized water and 0.077 mol of concentrated nitric acid (4.85 g) are added into a 1000 ml three-necks bottle comprising a stirrer and a controller. While stirring up, 0.017 mol silver peroxide (4.25 g) is added to the mixture solution until the silver peroxide completely dissolved. And then, 100 g of [NaZr₂(PO₄)₃] (zirconium sodium phosphate) is added to the mixture solution, and 20% of sodium hydroxide is used to adjust the mixture solution's pH value to a range 3-3.5. After then, the solution is heated to reach 60° C. to react for 6 hours. The mixture solution is then cooled at room temperature. Afterwards, the solution is filtered and the filter cake is rinsed until the pH value hit 6, and then the filter cake is dried at 120° C. for 1 hour and then be calcined at 900° C. for 2 hours, finally, the calcined substance is grinded by an air stream pulverizer to obtain powders having an average diameter 2.0 μm and 100 g of 3.7% in weight of silver-contained zirconium phosphate inorganic antibacterial agents.

### Embodiment 4

The preparing method is same with the example 3, wherein the 100 g [NaZr₂(PO₄)₃] (zirconium sodium phosphate) is replaced by A-type zeolite so as to obtain 100 g of 3.7% in weight of silver-contained inorganic antibacterial agents.

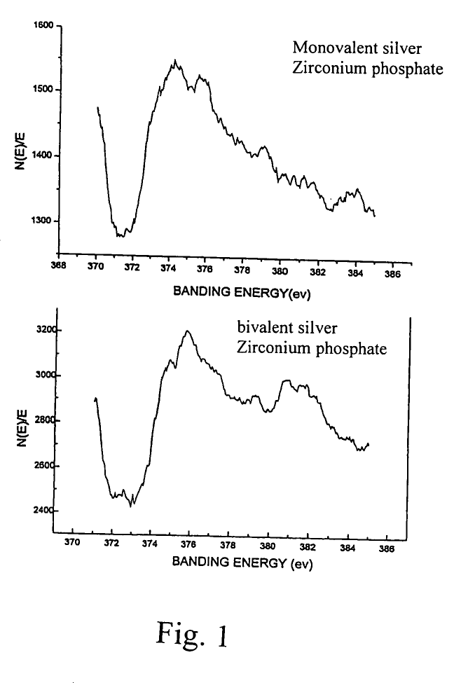

The XPS of FIG. 1 illustrates the difference between the bivalent silver contained zirconium phosphate prepared by the first example of the present invention and monovalent silver contained zirconium phosphate available on the market.

Antibacterial Performance Experiment

First of all, the bivalent silver contained zirconium phosphate prepared by the first example of the present invention and monovalent silver contained zirconium phosphate available on the market are prepared by a comparison.

First of all, 1% in weight of inorganic antibacterial agents sodium zirconium phosphate containing monovalent silver is thoroughly mixed with acrylate paint to be stirred up for at least 30 minutes until the paint and the antibacterial agent evenly mixed. And then, the mixed paint is sprayed onto a metal plate.

Secondly, 0.75% in weight of inorganic antibacterial agents sodium zirconium phosphate containing divalent silver of the present invention is thoroughly mixed with acrylate paint for 30 minutes. The mixture is sprayed onto a metal plate as well.

Antibacterial performance is detected to the two sample above. Tests are performed according to the 2002 “disinfection techniques standard-antibacterial test” enacted by China Health Department, that is to say, the tests are performed by covering with film.

The results are below:

Test Results

| | | After 0 | After 24 | |
|---|---|---|---|---|
| Antibacterial | | hour in | hours in | |
| Paint | Bacteria | contact | contact | Antibacterial |
| Sample | Sample | cfu/cm² | cfu/cm² | Performance % |
| Paint for | Escherichia coli | 7.8 × 10⁴ | <2 | >99.99 |
| added 1% | (ATCC 25922) | | | |
| Market | Staphyloccus | 1.75 × 10⁵ | 6 | 99.99 |
| available | aureus | | | |
| agents a | (ATCC 6538) | | | |
| Paint for | Escherichia coli | 7.8 × 10⁴ | <1 | >99.99 |
| added 0.75% | (ATCC 25922) | | | |
| Agents of | Staphyloccus | 1.75 × 10⁵ | 6 | 99.99 |
| present | aureus | | | |
| invention | ATCC 6538 | | | |

It is shown from the above table that inorganic antibacterial agents sodium zirconium phosphate prepared by the present invention has a better antibacterial performance than inorganic antibacterial agents sodium zirconium phosphate containing monovalent silver available in the market. There is no doubt that such antibacterial paint could be used on handle bars, computers, telephones, toys, or wood floors.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. An inorganic antibacterial agent containing high-valence silver, which are characterized by containing 2 to 6% by weight divalent silver, trivalent silver or tetravalent silver based on total weight of the antibacterial agents, wherein said high-valence silver is supported onto a solid carrier by an ion exchange reaction.

2. The inorganic antibacterial agent containing high-valence silver, as recited in claim 1, wherein said solid carrier is selected from a group consisting of sodium zirconium-phosphate, titanium phosphate, tin phosphate and zeolite.

3. The inorganic antibacterial agent containing high-valence silver, as recited in claim 2, wherein said zeolite is selected from a group consisting of A-type zeolite, X-type zeolite, and Y-type zeolite.

4. The inorganic antibacterial agent containing high-valence silver, as recited in claim 1, further containing 3.7% by weight of said divalent silver, said trivalent silver or said tetravalent silver based on a total weight of said antibacterial agent.

5. The inorganic antibacterial agent containing high-valence silver, as recited in claim 1, wherein an average particle diameter of said inorganic antibacterial agent is ranged from 1.0-10.0 μm, preferably 1.0-2.0 μm.

6. A method for preparing an inorganic antibacterial agent containing high-valence silver, comprising the following steps:
adding a solid carrier, which is capable of ion exchange, into a solution containing high-valence silver;
substantially stirring said solution to obtain a pulp formed solution for enabling an ion exchange reaction between said high-valence silver ion and the exchangeable ion of said solid carrier to yield a solid compound, and
filtering and drying said solid compound to ultimately obtain said inorganic antibacterial agent containing high valence silver.

7. The method, as recited in claim 6, wherein said solution containing high-valence silver is prepared by dissolving silver peroxide into persulphate or concentrated nitric acid to generate water solution containing bivalent sliver, periodic acid solution containing trivalent silver, and sliver acid solution containing tetravalent silver.

8. The method, as recited in claim 6, wherein said solid carrier is selected from a group consisting of sodium zirconium phosphate, titanium phosphate, tin phosphate and zeolite.

9. The method, as recited in claim 6, wherein a volume ratio between said solid carrier and said solution containing high-valence sliver is 1:6-10, preferably 1:8.

10. The method, as recited in claim 6, wherein said ion exchange reaction between said high-valence silver and said solid carrier is reacted at a predetermined condition, wherein a pH value is ranged 1-5, preferably 3-5, temperature ranged 30° C. to 80° C., preferably 55° C. to 65° C., best at 55° C., reacting time ranged 2-8 hours, preferably 4-6 hours, wherein 20% NaOH or KOH is applied for adjusting said pH value.

11. The method, as recited in claim 6, wherein said filtering and drying step further comprises sub-steps for washing a filter cake until a pH value ranged between 5-6, preferably 6, and for drying said filter cake at a temperature between 110° C.-140° C., preferably at 120° C. for 1-2 hours.

12. The method, as recited in claim 6, further comprising a step for calcinating said solid compound between 800° C. to 1000° C., preferable at 900° C., for 2-4 hours, preferable 2 hours, and a step for grinding said solid compound by a gas flow pulverizer to obtain particles with a size of average diameter of 1.0-10.0 μm, preferably 1.0-2.0 μm.

13. comprising antibacterial clothing, antibacterial daily products, antibacterial plastic products, antibacterial medical and mechanical devices, antibacterial structure materials, antibacterial ceramics, antibacterial sanitary ware, and antibacterial home electronic appliances.
